(19) United States
(12) Patent Application Publication
(10) Pub. No.: US 2003/0078558 A1
(43) Pub. Date: Apr. 24, 2003

(54) Disposable pant type absorbent article with improved fasteners and method of making same

(76) Inventors: Hamzeh Karami, Lockhaven, PA (US); Babak Damaghi, Kings Point, NY (US)

Correspondence Address: Stoll, Miskin, Hoffman & Badie, The Empire State Building, Suite 6110, 350 Fifth Avenue, New York, NY, 10118, US

(21) Appl. No.: 10/266,420

(22) Filed: Oct. 8, 2002

Related U.S. Application Data
(63) Continuation-in-part of application No. 09/965,381, filed on Sep. 27, 2001.
Application No. 09/965,381 is a continuation-in-part of application No. 09/844,726, filed on Apr. 27, 2001.
Application No. 09/844,726 is a continuation-in-part of application No. 09/797,334, filed on Mar. 1, 2001.
Application No. 09/797,334 is a continuation-in-part of application No. 09/247,629, filed on Feb. 10, 1999.

(51) Int. Cl. A61F 13/15

(52) U.S. Cl. 604/391

(57) ABSTRACT

An integral pull-up type absorbent article such as a diaper is provided which has improved hook and loop fasteners for more effective fluid leak prevention. The diaper comprises a coversheet, a backsheet, a waist region having a front waist portion and a back waist portion each having a lateral portion terminating at a lateral edge, a strip of loop material at each lateral edge of the front waist portion and a tape tab with a folded portion having a hook surface at each lateral edge of the back waist portion, wherein when said back waist portion is folded over said front waist portion, each of the hook material engages a correspondingly aligned loop strip. In one embodiment the outer surface of the front waist region may itself be made of loop material for engagement with the hook fastener.

A method is also provided for making the present pull-up type absorbent articles.

### RELATED APPLICATIONS

This application is a continuation in part of commonly assigned, copending application Ser. No. 09/965,381 filed Sep. 27, 2001, which is, in turn, a continuation-in-part of application Ser. No. 09/844,726 filed Apr. 27, 2001, which is, in turn, a continuation-in-part of application Ser. No. 09/797,334 filed Mar. 1, 2001, which is, in turn, a continuation-in-part of application Ser. No. 09/247,629 filed Feb. 10, 1999.

### FIELD OF THE INVENTION

The present invention relates generally to disposable absorbent articles such as training pants, diapers, undergarments and incontinent briefs, and is more particularly related to such pants, diapers, undergarments and briefs which have become known in the art as “pull-up” or “pull-on” absorbent articles. In one particular aspect, the present invention relates to an absorbent article of the aforementioned types which, due to its unique construction and improved fasteners, assures fitness and comfort to the wearer, protects against leakage of fluids and other body exudates and can be readily opened for inspection and removal from the wearer. In another aspect this invention also relates to ready-to-wear pull-up type absorbent articles.

The present invention also relates to a method of producing such absorbent articles.

### BACKGROUND OF THE INVENTION

Disposable absorbent articles such as disposable baby diapers and adult incontinent briefs, underpants, guards and the like articles are widely used in homes and in various health care facilities and institutions. Indeed the use of such articles has become a common sanitary practice, and while initially such absorbent articles were used mostly for infant care, more recently their use has been expanded to include adults as well. In both instances, the absorbent article must be designed to effectively prevent leakage of urine and other fecal materials, while insuring body fit and comfort.

Most presently available absorbent articles are generally unitary in structure, pre-shaped and pre-folded, and comprise an absorbent pad having a liquid permeable top sheet facing the wearer's body, a liquid impermeable backsheet on the opposite side, and an absorbent sheet or panel disposed between the top sheet and the back sheet. The absorbent article comprises a front side portion, a crotch portion and a backside portion, and further includes elastic members along the circumference of the waist and around the leg openings. While the heretofore commercially available absorbent articles have been somewhat effective against leakage of body fluids and fecal materials, and have therefore met some degree of acceptability, they have not been entirely satisfactory for their intended applications. In other words, they have not proven to be entirely leak proof, nor have they completely prevented issuance of the body exudates outside the diaper or the underpants. These deficiencies are primarily due to inadequate and loose body fit, which result in leakage of the body fluids and solids through the legs' openings. These problems are even more pronounced in case of adults because of their diverse body shapes and varying contours. Another disadvantage of the commercially available absorbent articles such as diapers, incontinent briefs and the like, is associated with opening and removing the soiled article for inspection without soiling the wearer's leg or body, or changing the diaper while the wearer has his or her shoes and pants on.

There are several patents which disclose various attempts made in the prior art over the past years to eliminate, or at least minimize, the shortcomings of the present commercially available absorbent briefs. Some of these patents are referred to in the aforementioned commonly assigned, copending application Ser. No. 09/965,381. That patent application describes a pull-up diaper comprising a coversheet, a backsheet, an absorbent layer disposed between the coversheet and the backsheet, a front waist region, a back waist region, a crotch region and a pair of leg openings through which extends the legs of the wearer of the diaper. The front and back waist regions are provided with fasteners for fastening the two regions together. In one embodiment, the fastening system comprises a pair of loop strips located that the lateral edges of the front waist region and a pair of correspondingly aligned hook strips located at the lateral edges of the back waist region such that when the back waist region and the front waist region are overlapped during wear, each hook strip releasably engages a correspondingly aligned loop strip. Other fastening systems are disclosed and in one variation the back waist region comprises one or more tape tabs located at its lateral edges. Each tab has one surface attached to the back waist region and an opposed hook surface aligned with a loop strip in the waist region. In order to fasten the diaper such as during wear, the back waist region and the front waist region are overlapped so as to engage the hook surface of each tab with a correspondingly aligned loop strip on the front waist region.

In a recent patent, i.e., U.S. Pat. No. 6,027,484 issued Feb. 22, 2000 to Anette Remare, a pant diaper is described comprising a piece of fibrous nonwoven or plastic elastic material 9 having two parts 13 and 14 which can be pulled apart to define the side parts or flaps of the diaper. The side parts are fastened together by means of the hooks 15,16 and the loop 17 as shown in FIGS. 1 and 2. The piece 9 is joined to the outer casing 3 of the front part of the diaper by the glue points 10 which may be homogeneously distributed as shown in FIG. 1 or non-homogenous glue points or fastening means 110 as shown in FIG. 3.

A more recent patent, i.e., U.S. Pat. No. 6,287,287 B1 issued Sep. 11, 2002 to Laura Linda Elsberg describes a prefastened disposable article which includes a pair of primary fasteners located on opposed side edges of one waist region. The primary fasteners overlap and releasably engage the opposite waist region. A pair of passive bonds releasably connect the overlapped portion of one waist portion to the opposite waist region in order to maintain the article in prefastened condition.

In general, the briefs and diapers described in the prior art patents have a common structural deficiency in that they are provided with side seams which are welded together by heat and pressure or vibration (ultrasonic welding). Side seals must be sufficiently strong to hold the diaper on the person and must be capable of being torn so that the wearer can tear it easily in order to inspect or change the diaper while having his or her shoes on. Diapers having hook and loop fastening systems as described in the aforementioned copending application Ser. No. 09/965,3812 provide improvements over the prior art diaper, but nevertheless there is still a need for pull up type absorbent briefs and diapers which are comfortable to wear, highly effective against leakage of fluids and feces, can be readily inspected for soil and which have compact fastener that is folded in place when the diaper is not being used.

Accordingly, it is an object of the present invention to provide a pull-up type disposable absorbent article such as infant diapers, adult incontinent underpants, briefs, guards and the like articles, which overcome the deficiencies and shortcomings of the prior art absorbent articles.

It is another object of this invention to provide disposable absorbent articles which, due to their unique construction, provide improved fit to the body and prevent leakage of urine and other body exudates through the leg openings, and which are easy to take apart for inspection.

It is also an object of this invention to provide such disposable articles which utilizes a unique hook and loop fastening system in order to assure leakage prevention, simplify opening, inspection and reassembling the diaper after inspection, and which is comfortable to wear by incontinent persons.

It is an additional object of this invention to provide a method of making such absorbent articles.

The foregoing and other objects and features of the present invention will be more fully comprehended and appreciated from the ensuing detailed description and drawings which form parts of this application.

It must be understood throughout this application that the term “pull-up” as used herein is synonymous with “pull-on” as used in the prior art patents.

### SUMMARY OF THE INVENTION

In order to achieve the foregoing objects and desirable features, the present invention provides a disposable absorbent article such as pull-up diaper which comprises a liquid permeable coversheet, a liquid, air and vapor impermeable backsheet, an absorbent core or layer, made of fiberized wood pulp containing superabsorbent polymer (SAP) disposed between the coversheet and the backsheet, an outer layer of air and liquid permeable spunbond nonwoven polypropylene and an inner layer of air and liquid permeable spunbond polypropylene. The diaper also comprises elasticated crotch region having elastics on each side of the absorbent layer such that none of the “active length” of the elastic bands intersects the thigh elastic. The term “active length” refers to the length of the elastic band which is attached on the insert sides, under tension. A contoured insert containing the absorbent core is sandwiched between the coversheet and the backsheet. Side seals are formed by welding several layers of polypropylene spunbond nonwoven together.

The diaper is provided with hook and loop fasteners. A pair of loop strips are attached at the lateral edges of front waist portion of the diaper and a pair of fastener tabs with a hook surface are attached at the lateral edges of the back waist portion of the diaper. Each fastener tape has a portion which is permanently attached to the outer surface of the back waist and a portion which is releasably attached to that surface.

A strip of hook material is secured to the fastener side of the tape and a portion of the fastener is folded under the permanently attached portion of the tape and the fold is kept in place by an adhesive or heat spot. When the diaper is worn, the back waist portion is folded onto the front waist portion and each of said hooks engages a complementary loop surface to close the diaper.

The method of attachment of hook and loop fasteners to the diaper of this invention comprises first separately attaching individual precut hook strips to the lateral edges of the outer surface of the front waist portion of the diaper chassis. Thereafter, the diaper chassis is folded and precut loop strips are attached to the lateral edges of the outer surface of the back waist portion of the diaper chassis. The hook and loop strips can be attached to the diaper chassis in a continuous operation as the chassis travels in one direction, usually the machine direction.

Alternatively, the outer surface of the front waist portion of the chassis may be made of loop material, in which case the hook strips adhere directly to the outer surface of the front waist portion. This eliminates the method step of attaching loop strips as hereinbefore described.

### BRIEF DESCRIPTION OF THE DRAWINGS

In the drawings, wherein like: reference numerals are employed to designate like parts wherein possible:

FIG. 1 is a perspective view of the disposable absorbent article of the present invention shown as a pull-up diaper having side seals which are broken apart on one side for illustrative purposes;

FIG. 2 is a stretched plan view of the pull-up diaper shown in FIG. 1;

FIG. 3 is a sectional view taken along the line 3-3 of FIG. 2;

FIG. 4 is a sectional view taken along the line 4-4 of FIG. 2;

FIG. 5 is a schematic view illustrating overlapping of the front waist region and back waist region of the diaper shown in FIG. 1 and the manner of their attachment by hook and loop;

FIG. 6 is a stretched plan view similar to FIG. 2 but illustrating another variation of hook and loop fasteners;

FIG. 7 is a schematic view similar to FIG. 5 but illustrating the hook and loop fastener arrangement in FIG. 6;

FIG. 8 is a view similar to FIG. 2 but using tape tabs with hooks in combination with loop fasteners wherein the tape tabs are located on the inside surface of the back waist;

FIG. 8A is a cross-sectional view taken along the line 8A-8A of FIG. 8;

FIG. 8B is a cross-sectional view taken along the line 8B-8B of FIG. 8;

FIG. 8C is a view similar to FIG. 5 showing the overlapping of the front waist and the back waist of the diaper;

FIG. 9 is a perspective view of the diaper shown in FIG. 8 with the tape tabs pulled away from the loop fasteners and the diaper ready to wear;

FIG. 10 is a stretched plan view of a pull-up diaper according to another embodiment of the present invention wherein the tape tabs are located on the outer surface of the back waist;

FIG. 10A is a cross-sectional view taken along the line 10A-10A of FIG. 10;

FIG. 10B is a cross-sectional view taken along the line 10B-10B of FIG. 10;

FIG. 10C is a view similar to FIG. 8C but showing the manner of fastening the front waist and back waist of the diaper shown in FIG. 10;

FIG. 11 is a stretched plan view of a different embodiment of the present invention similar to the embodiment illustrated in FIG. 10 with the tape tabs located on the outside surface of the back waist region, folded and adhesively secured to said surface;

FIG. 11A is a cross-sectional view taken along the lines 11A-11A of FIG. 11;

FIG. 11B is a cross-sectional view taken along the line 11B-11B of FIG. 11;

FIG. 11C is a view similar to FIG. 10C showing the overlapping the front waist and back waist of the diaper;

FIG. 12 is a stretched plan view of a different embodiment of the present invention similar to the embodiment illustrated in FIG. 10 with the tape tabs located on the outside surface of the back waist region and projecting outside of the lateral edges;

FIG. 12A is a cross-sectional view taken along the line 12A-12A of FIG. 12;

FIG. 12B is a cross-sectional view taken along the line 12B-12B of FIG. 12;

FIG. 12C is a view similar to FIG. 10C showing fastening system;

FIG. 13 is a stretched plan view of still another embodiment of the invention similar to FIG. 12 but having a weakened perforated line adjacent the side seal;

FIG. 14 is a view similar to FIG. 8 but illustrating the improved hook and loop arrangement in accordance with the present invention;

FIG. 15 is a sectional view taken along the line 15-15 of FIG. 14 showing the manner of attachment the tape tab with a hook surface to the back waist portion of the diaper;

FIGS. 15A, 15B, 15C, 15D, 15E and 15F each represents a schematic diagram of different arrangement of the hook surface, at different positions;

FIG. 15G is similar to FIG. 15 but illustrates an alternate fastener construction.

FIG. 16 is a sectional view taken along the line 16-16 of FIG. 14, and FIGS. 17-19 are schematic representations illustrating the method of attachment of the hook and loop fasteners to the diapers in accordance with the present invention.

### DETAILED DESCRIPTION OF THE INVENTION

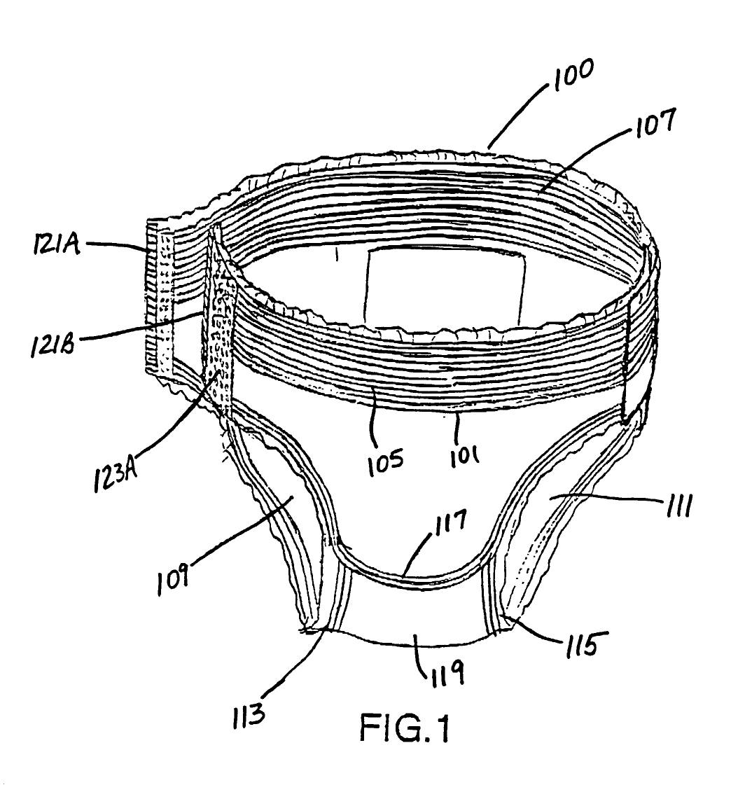

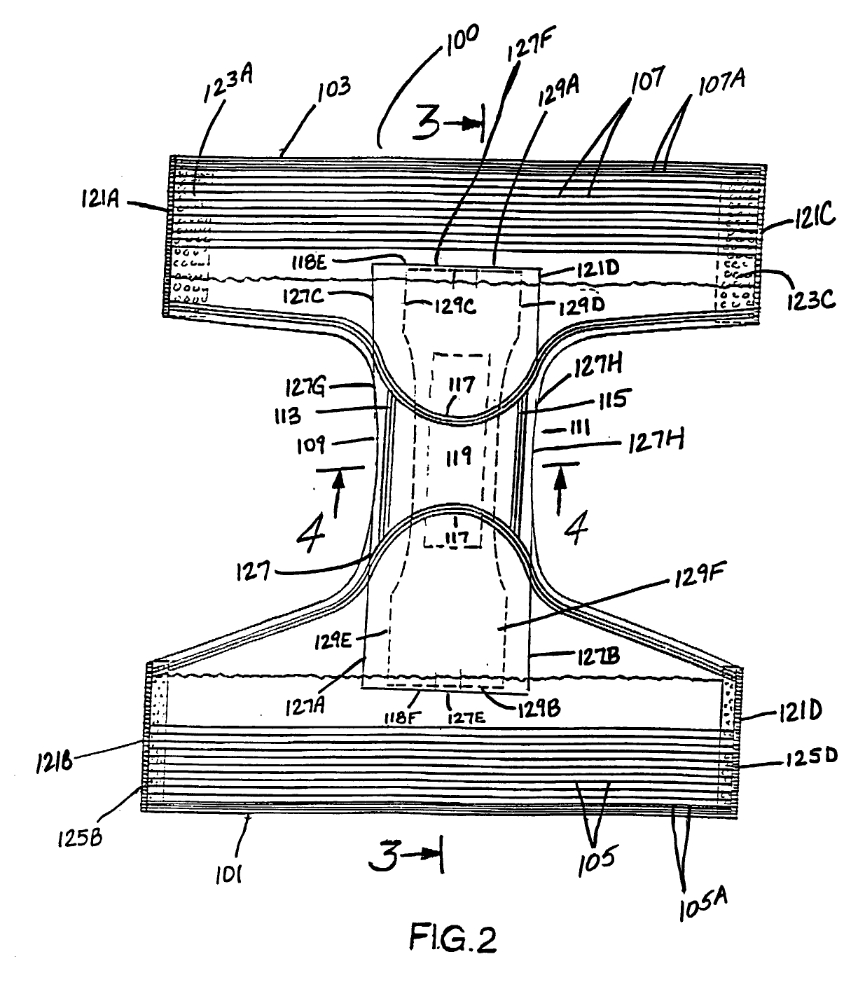

Referring to FIG. 1, there is shown a pull-up diaper generally designated as 100 comprising an elasticated back waist region 101 and an elasticated front waist portion 103. The elasticated back waist region 101 includes an elastic band comprising a plurality of elastic members 105 and belly elastics 105A spanned across the back waist 101, and the elasticated front waist region 103 includes an elastic band comprising a plurality of elastic members 107 and belly elastics 107A spanned across the front waist 103. The diaper 100 also comprises a pair of elasticated leg openings, i.e., a right elasticated leg opening 109 and a left elasticated leg opening 111. The left leg opening 109 is provided at its peripheral edges with the crotch elastic members 113 and the right leg opening 111 is also provided at its peripheral edges with the crotch elastic member 115. Each leg opening also comprises a thigh elastic member 117 which is usually tensioned from about 0 to about 400 percent elongation, preferably from about 150 to about 250 percent elongation. The peripheral crotch elastic members 113 and 115 may also tensioned from about 0 to about 400 percent elongation, preferably from about 200 to about 300 percent elongation so that the leg openings fit snugly against the crotch region 119 of the wearer in order to prevent leakage of urine or other body exudates through the leg openings. The front and back of the diaper 100 are provided with the side seals 121A,121B disposed at the outer left edges of the back waist region 101 and the front waist region 103, and similar side seals 121C and 121D are disposed at the outer right edges of the back waist region 101 and the front waist region 103 as shown in FIG. 2. These side seals may be formed by heat, pressure, combination of heat and pressure, or by a suitable adhesive in a manner known in the prior art. The side seals preferably have low peel strength so that when the seals are torn or peeled away their external edges remain clean. Preferably, the side seals strength may be from 1 to about 3 pounds per inch, and more preferably less than about 1 pound per inch.

The diaper may also be provided with elastics at the belly portion in the front or back but such belly elastics are not strictly necessary for some diapers.

Ordinarily, in order to inspect the diaper during wear, the seal is ripped open and the diaper is inspected for the presence of feces or exudates. Once inspected, the diaper is disposed of since it is often difficult to effectively reseal the side seals. In the embodiment of the present invention shown in FIG. 2, the diaper is provided with two strips of loop material 123A, 123C disposed adjacent the side seals 121A and 121C. Both the side seals and the strips of the loop fastener material are disposed parallel to the vertical axis of the diaper. Similarly, strips of hook material 125B, 125D are disposed adjacent the side seals 121B,121D, parallel to the vertical axis of the diaper. During use, when the front and back portions of the diaper are folded, the hook strips 125B and 125D engage onto the loop strips 123A and 123C, respectively thus providing additional sealed regions at the lateral edges of the front waist portion and the back waist portion. This construction permits opening the diaper for inspection by disengaging the hook and loop strips 123A and 125B, or the hook and loop strips 123C and 125D in order to inspect the diaper. If no feces or exudates are found, the diaper is closed, i.e., resealed by re-engaging the hook and loop strips without disposing of the diaper. FIG. 1 shows the diaper during wear with one edge partially open and the hook and loop strips in disengaged positions. It is preferable that the loop strips be located inside surface the hook be located on the outer surface of the diaper.

Referring again to the drawings, more specifically to FIGS. 2-5, the diaper 100 comprises an insert member 127 which contains the absorbent core 129 sandwiched between the cover or top layer or sheet 131 (facing the body of the wearer) and the polyethylene backing film 133. The insert 127 is secured, adhesively or by some other suitable means, to a spunbond nonwoven layer 135. Optionally, the absorbent core 129 may be covered by the bottom tissue layers 137 generally made of wood pulp fibers or similar material. An acquisition layer 139 is interposed between the cover sheet 131 and the core layer 129 and serves to temporarily retain the body exudates and slowly distribute them through the absorbent core 129 in order to keep the skin dryer. The various layers are generally coextensive with one another and are sealed together to form a sealed composite structure.

As shown in FIG. 2, the absorbent core 129 spans substantial part of the length of the diaper 100 terminating at the front edge 129A, the back edge 129B, the right side edges 129C, 129D, and left side edges 129 and 129F. However, as it can also be seen from this figure, the back edge 129A and the front edge 129B of the absorbent core 129 are spaced apart a finite distance, which may be varied, relative to the diaper. The insert 127 is defined by the longitudinal side edges 127A, 127B, 127C and 127D, the lateral edges 127E and 127F, and includes the necked down region defined by the necked down contoured side edges 127G and 127H. The necked down region defined by the necked down side edges is elasticated at both sides by the elastic members 113,115. Three elastic members are shown although fewer or greater numbers of elastic members may be used if desired.

As previously mentioned, each leg opening 109, 111 is tensioned by a thigh elastic member 117 shown as a curved elastic in FIG. 2, but may be straight elastic element if desired. The thigh elastic may be tensioned from about 0 to about 400 percent elongation, preferably from about 150 to about 300 percent elongation for more improved fitness around the legs.

As is further shown in FIG. 2, the diaper 100 of the present invention has an elasticized crotch region 119 which is provided with one or more spaced-apart right elastic members 113 disposed interiorly of the leg right opening 109 on the right side edge of the insert, and one or more spaced-apart left elastic members 115 disposed interiorly of the leg opening 111 of the left side of the insert 127.

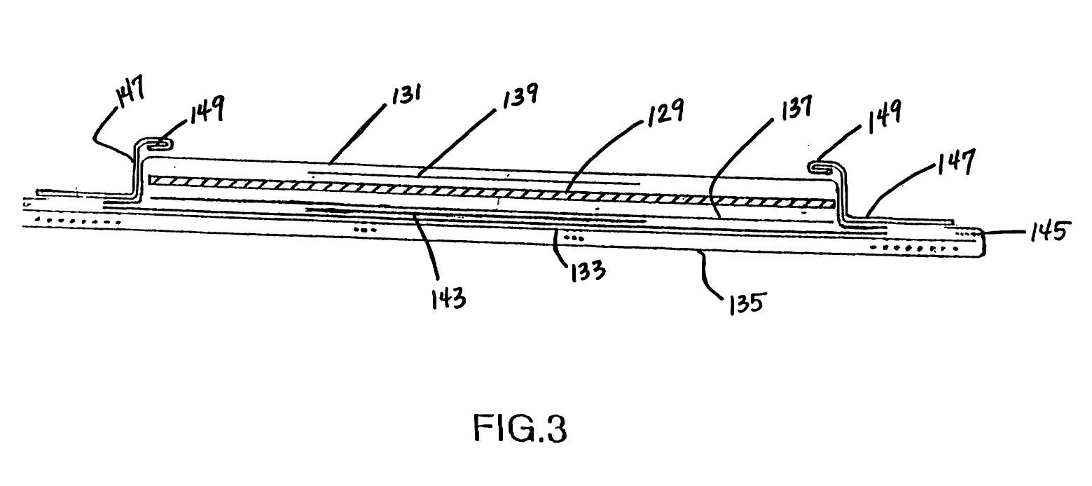

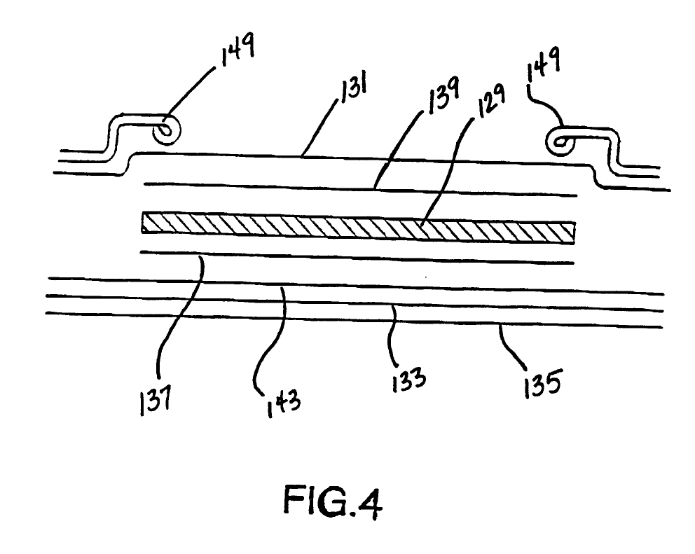

Referring to FIGS. 3 and 4, the coversheet or layer 131 is conveniently made of spunbond nonwoven polypropylene which is available from First Quality Fibers, Inc., McElhattan, Pa. The acquisition layer 139 is usually made of chemically bonded nonwoven polypropylene available from American Nonwovens, Columbus, Mo. Preferably, the width of this layer is substantially the same as the width of the absorbent core 129. This core may be made pulp fibers and superabsorbent polymers such as IM 7000 series available from Clariant Products, Inc., Portsmouth, Va., and Chemdal 200 series, available from Chemdal, Inc., Palantine, Ill. Alternatively, the absorbent core 129 may be made of dual layer construction, in which case, the absorbent polymer may be securely positioned between each layer of the absorbent material.

The film backing 133 is usually a polyethylene layer which is liquid, air and preferably vapor impermeable, and is placed under the absorbent core member 129 to prevent body exudates from leaking and otherwise soiling the user's bed and clothing. The width and length of the backing film 133 are generally at least equal to the width and length of the absorbent core 129. Polyethylenes suitable as backing film for the purpose of this invention are available from Clopay Plastics, Cincinnati, Ohio as is further shown in FIGS. 3 and 4, a layer 135 of spunbond nonwoven polypropylene is disposed as a backing layer and covers the area under the insert 127. This layer is usually coextensive with the overall width and length of the pull-up diaper.

As is further shown in FIGS. 2 and 3, there is one elasticated crotch cuff 143 on each side of the garment. Each of these crotch cuffs is formed of a layer of spunbond nonwoven polypropylene laminated by hot-melt adhesive or by heat, and forms a fluid and an air impermeable composite structure. The crotch cuffs are under no tension or are tensioned from about 100 to about 200 percent so that the garment can fit snugly against the body and prevent leakage of body fluids of exudates, without pinching the body of the wearer. Additionally, these cuffs act as barriers against fluid leakage on each side of the absorbent core.

The garment of the present invention also has an elasticated waist cuff 145 which, similar to crotch cuffs, is not tensioned or is minimally tensioned between about 1 to about 100 percent elongation in order to provide a tight body fit which is leak-proof without pinching the body or causing discomfort to the wearer.

The insert 127 is adhesively secured to the nonwoven backing film 133 and, as shown in FIG. 2, the crotch width of the insert 127 is narrower than its width at the waist. As previously mentioned, there are usually three elastic members 113,115 on each side, although fewer or more elastic members can be used, as desired.

As is further shown in FIG. 3, the pull-on diaper of this invention has a waist cuff base 147 with a waist cuff apex 149 on both the front and back of the article. Similar to the crotch cuffs, these waist cuffs prevent fluid leakage from the ends of the core members 129.

In the embodiment shown in FIG. 2, the hook and loop fastener strips are located adjacent the side seals. However, in a variation of this embodiment, the hook and loop fastener strips may be used without the side seals and put on the diaper without the wearer taking of his or her shoes or pants. When in use, the wearer may disengage the strips, inspect the diaper for leaks and/or exudates and if free from such materials, the hook and loop strips may be refastened. The hook and loop fasteners may be attached to the inside or outside of the diaper. However, it is preferable to attach the loops on the inside surface and the hooks on the outside surface since the hook material has a rough surface which would irritate the skin. If the diaper is provided with side seals, this seal may be torn and the hook and loop fasteners are used to fasten the diaper.

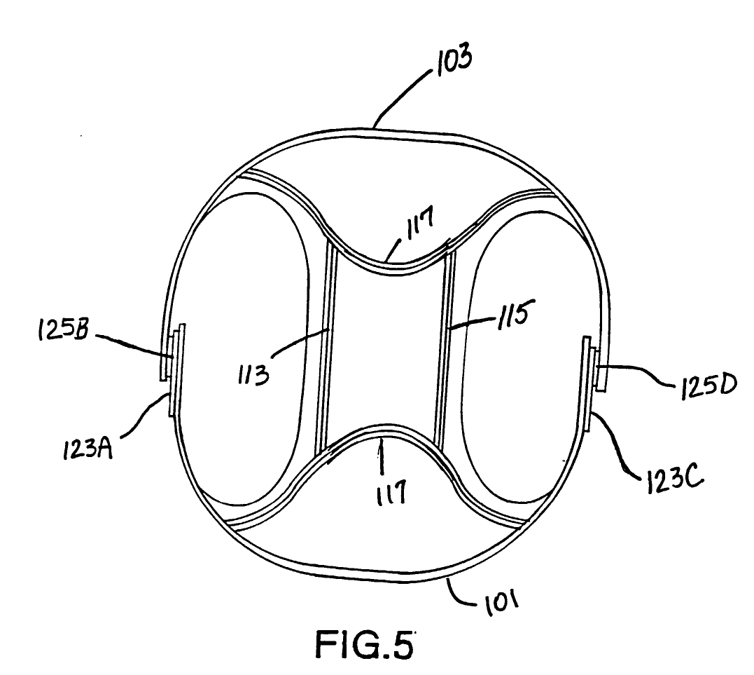

Another variation of the fastening system for the diaper of this invention is shown in FIG. 6. The basic components of the diaper in this figure is the same as the diaper shown in FIG. 2 except for the number and location of the hook and loop strips. Thus, referring to FIG. 6, the front and back of the diaper 200 are provided with the side seals 221A, 221B disposed at the edges of the front waist region 203, and side seals 221C and 221D are located at the outer left edges of the back waist region 201. These side seals may be formed by heat, pressure, combination of heat and pressure, or by a suitable adhesive in a manner known in the prior art. The back waist region 201 comprises a pair strips 205,207 of a loop material, with the loop strip 205 spaced inward relative to the edge or side seal 221D on the inner surface of the back waist region and the loop strip 207 spaced inward relative to the edge or side seal 221C or the outer surface of the back waist region. The term “strip” as used herein is not limited to any particular configuration as it may be rectangular, square, circular or any other shape and may be a patch or a section of the surface of material itself. Thus, the material itself may constitute a loop suitable for engagement with the hook strips. The front waist region 203 comprises the loop strip 209 spaced apart relative to the edge or side seal 221B on the outer surface and a hook strip 211 on the inner surface adjacent the loop strip 209 and separated therefrom by a weakened tear line such as the perforated line 213. The front waist region 203 also comprises a pair of side-by-side hook strips 215,217 spaced inward relative to the edge or side seal 221A, and separated from each other by a weakened tear line such as the perforated line 219. Both hook strips 217,215 are located on the inner surface of the front waist region 203. In order to assemble and fasten the diaper, when the perforated lines 213 and 219 are torn and the waist region 201 and waist region 203 are folded on each other, the loop strip 209 engages the hook strip 215 and the hook strips 211 and 217 engage the loop strips 205 and 207, respectively. A segment of a nonwoven material or some other suitable material may be used as enforcement or backup portion for the perforated lines in order to assure a clean tear of the perforated line.

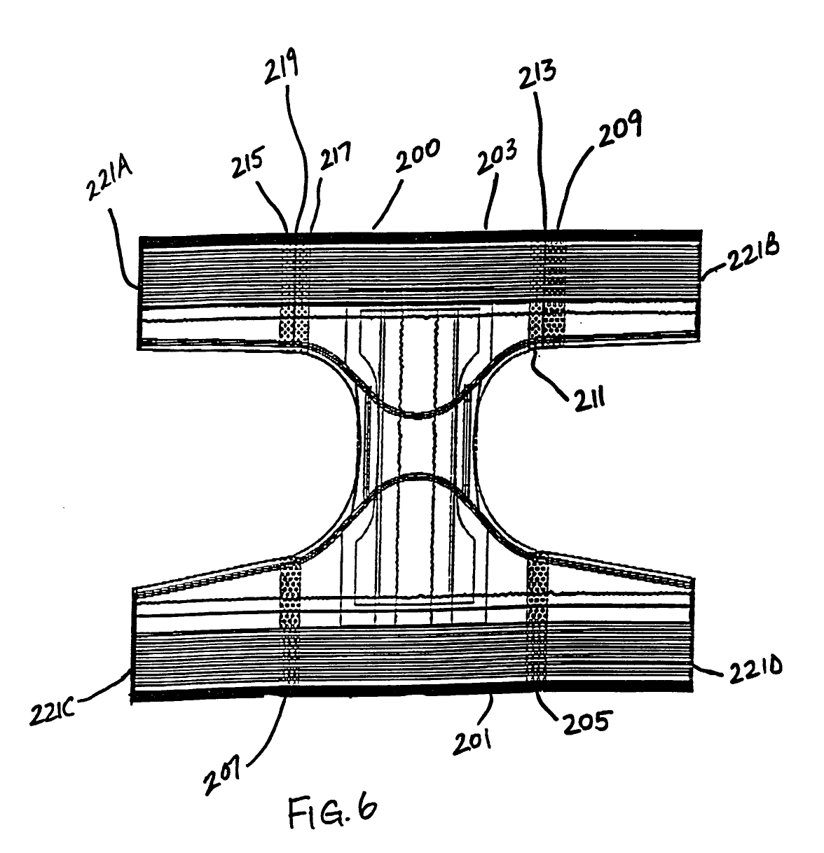

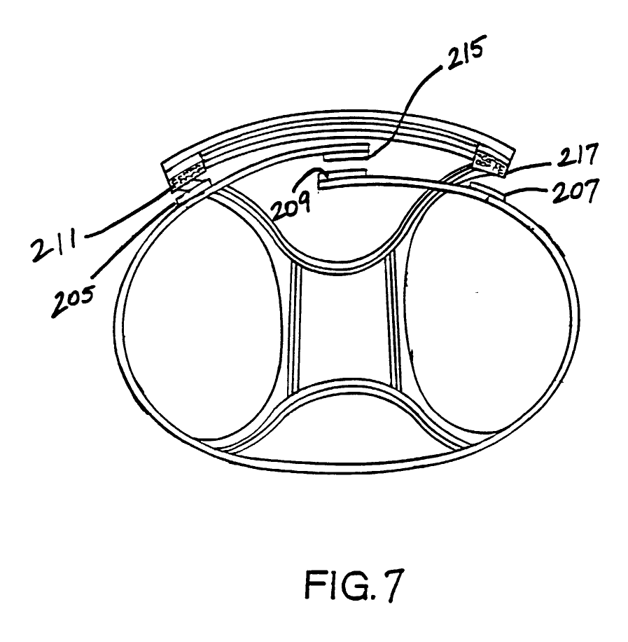

FIG. 7 is a schematic representation of the manner of fastening the hook and loop system shown in FIG. 6.

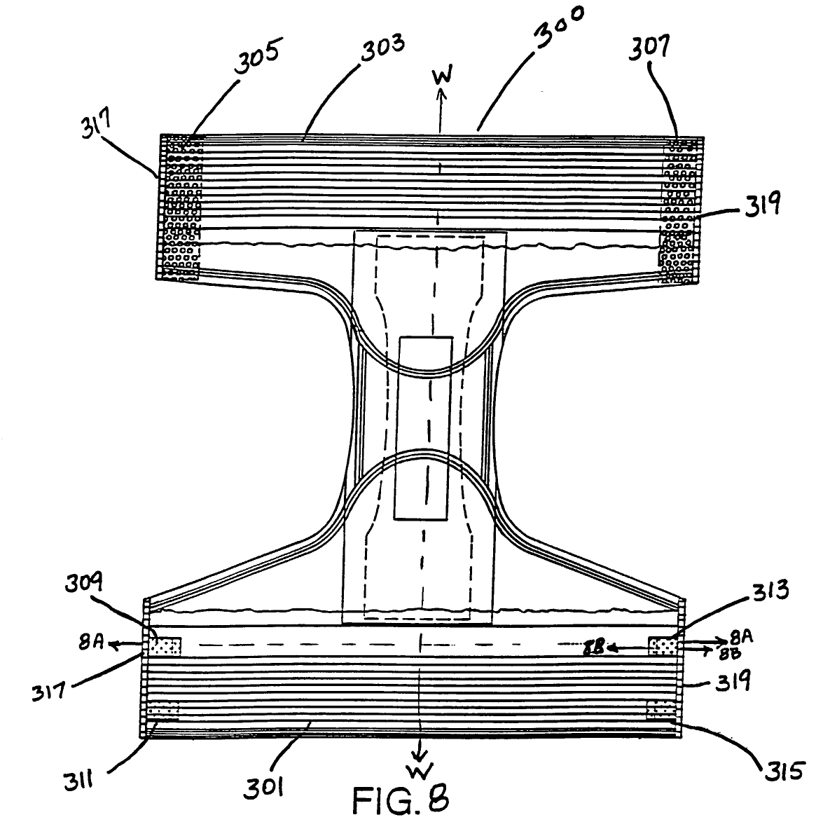

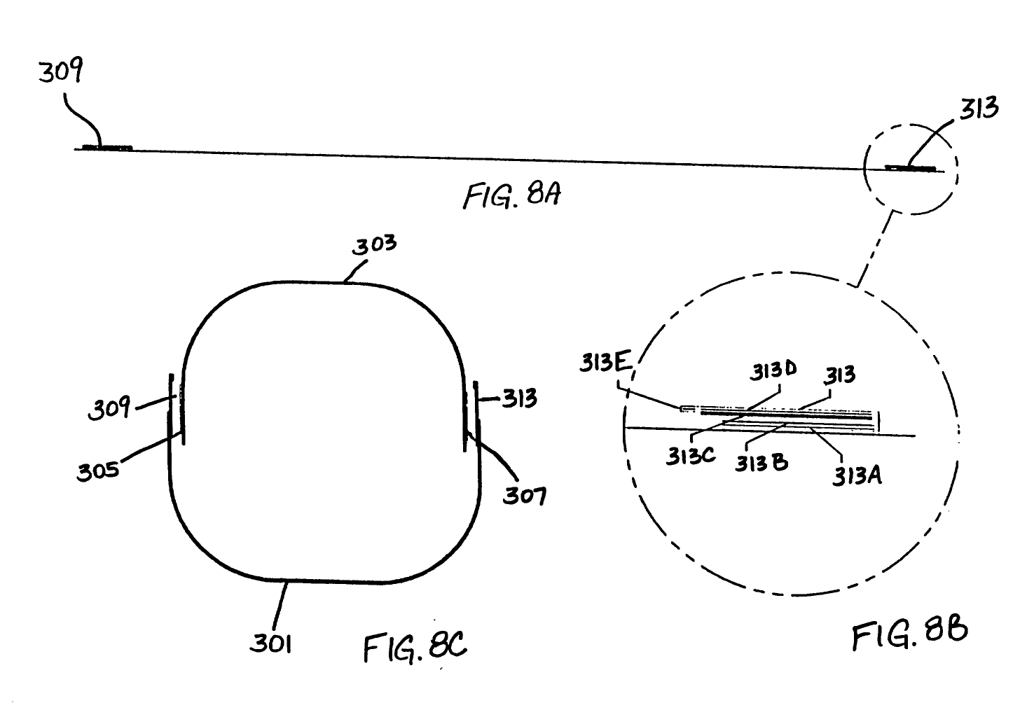

FIGS. 8-13 illustrate those embodiments of the invention using tape tabs as the male components of the fastening system. Otherwise, the structure of the diaper in these embodiments is the same as in FIGS. 2 and 6.

Thus referring to FIG. 8 the diaper shown therein is generally designated as 300 comprising a back waist region 301 having opposed lateral wings, and a front waist region 303 having similar opposed lateral wings, relative to the longitudinal axis W-W of the diaper. The front waist region 303 comprises a pair of strips 305,307 of loop material disposed on the outer surface near or at the lateral edge of the respective wings, and the back waist region 301 has tape tabs 309, 311, 313 and 315 attached thereto on the inside surface at or near the edge of the wings. As shown in FIGS. 8A and 8B the tape tab 313 has an adherent surface 313A attached to the back waist portion, a release paper 313B, a hook surface 313C opposite said adherent surface, and an adherent surface 313D for attaching said hook surface to the back waist region. The tape tab 313 has a finger lift 313E in order to expose the hook surface. The release paper 313B and the finger lift 313E are optional and not strictly necessary.

In order to fasten the diaper the side seals 317,319 are torn and the tape 313D is pulled away by lifting and pulling the finger lift 313E, the front waist region and the back waist region are then overlapped thereby engaging the hooks 309, 311, 313 and 315 onto the corresponding aligned loop strips 305 and 307. In the embodiment illustrated in FIG. 8 the front waist portion comprises the edge seal 317,319 at each lateral edge of the front waist region, and edge seals 321,323 at the lateral edges of the back waist region.

FIG. 9 is a perspective view of the pull-up diaper shown in FIG. 8 and is similar to the diaper shown in FIG. 2 except for the provision of the tape tabs having hook surfaces. Otherwise, the structures of the two diapers are the same.

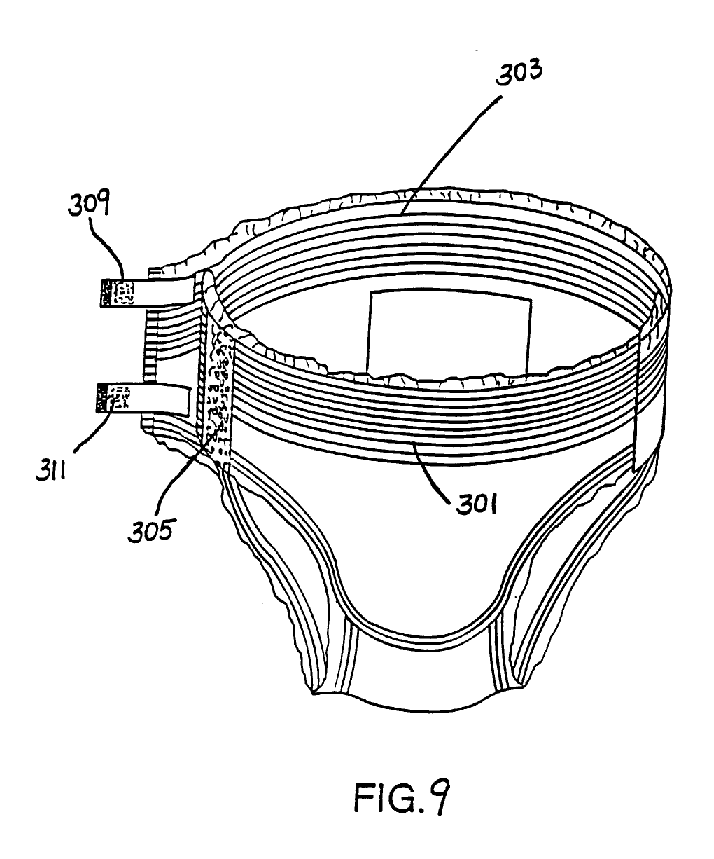

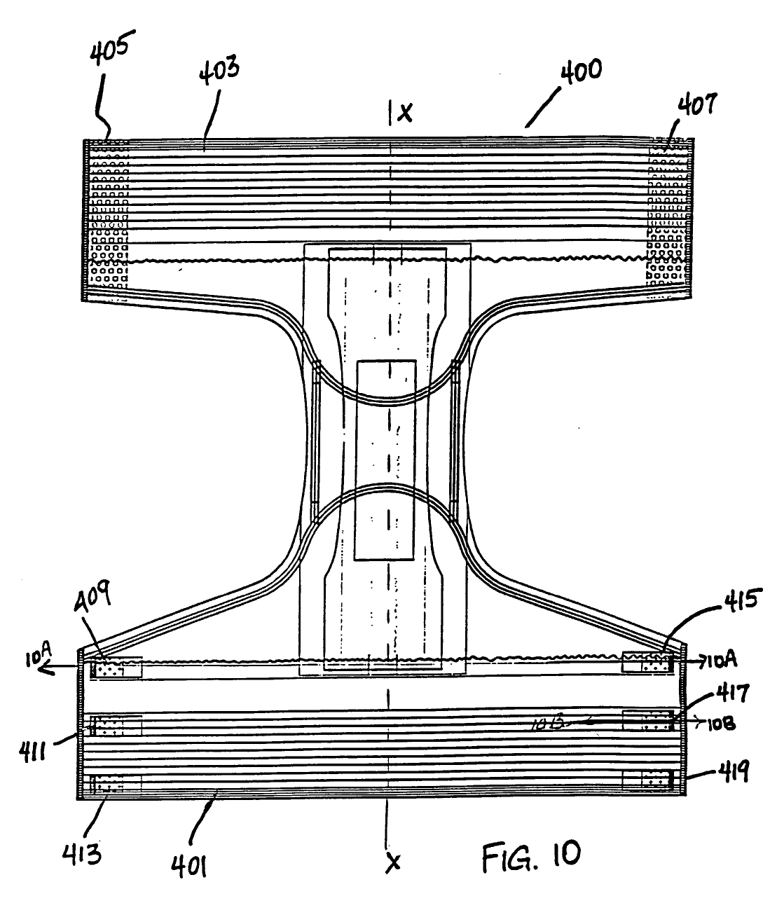

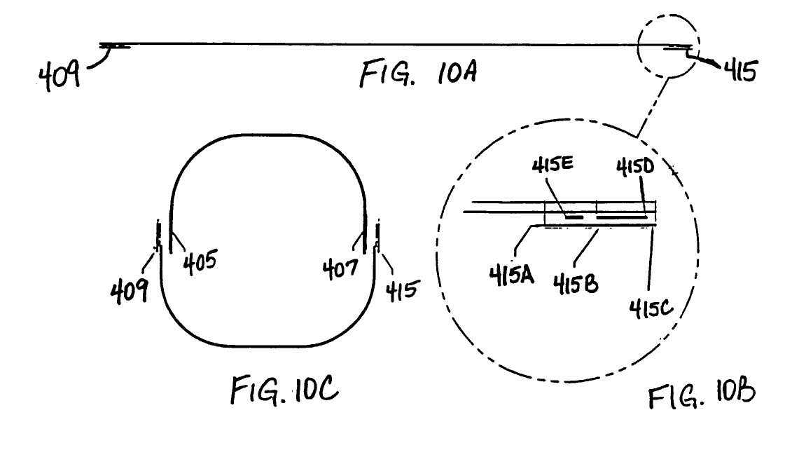

FIG. 10 is a stretched plan view of another embodiment of the invention similar to FIG. 8 but wherein the back waist region comprises three spaced apart tabs near the edge seal at each wing. Otherwise, the construction of the diaper is similar to the diaper shown in FIG. 8. Thus, the diaper shown in FIG. 10 is generally designated as 400 comprising a back waist region 401 having opposed lateral wings, and a front waist region having similar opposed lateral wings, relative to the longitudinal axis X-X of the diaper. The front waist region 403 comprises a pair of strips 405,407 of loop material, each strip being disposed near or at the lateral edge of its respective wing, and the back waist region 401 has three tape tabs 409, 411, 413 attached thereto near the edge of one of said wings, and three tape tabs 415, 417, 419 attached near the other wings. As shown in FIG. 10, the external edges of the tape tabs are spaced inwardly relative to the edge of each wing. Each of the tape tabs is attached to the outside surface of the back waist region 401. Thus, referring to FIGS. 10A-10C, tape tab 415 has an adherent surface 415A and a backing film 415B for attaching the tape tab to the back waist region 401. A finger lift portion 415C permits lifting the backing film away from the waist's outer surface. The tape tab has a hook surface 415D and a release paper 415E. The remaining tape tabs, i.e., tape tabs 409, 411, 413, 417 and 419 have a structure similar to tape tab 415 and are positioned on the outer surface of the back waist region in the same manner. These tapes are attached to the back waist surface such that each finger lift edge is adjacent to the side seals.

The back waist region 401 and the front waist region 403 are fastened together in the same manner described in connection with the diaper shown in FIG. 8. Also, shown in FIG. 10, the front and back waist regions comprise edge seals at each lateral edge or wing.

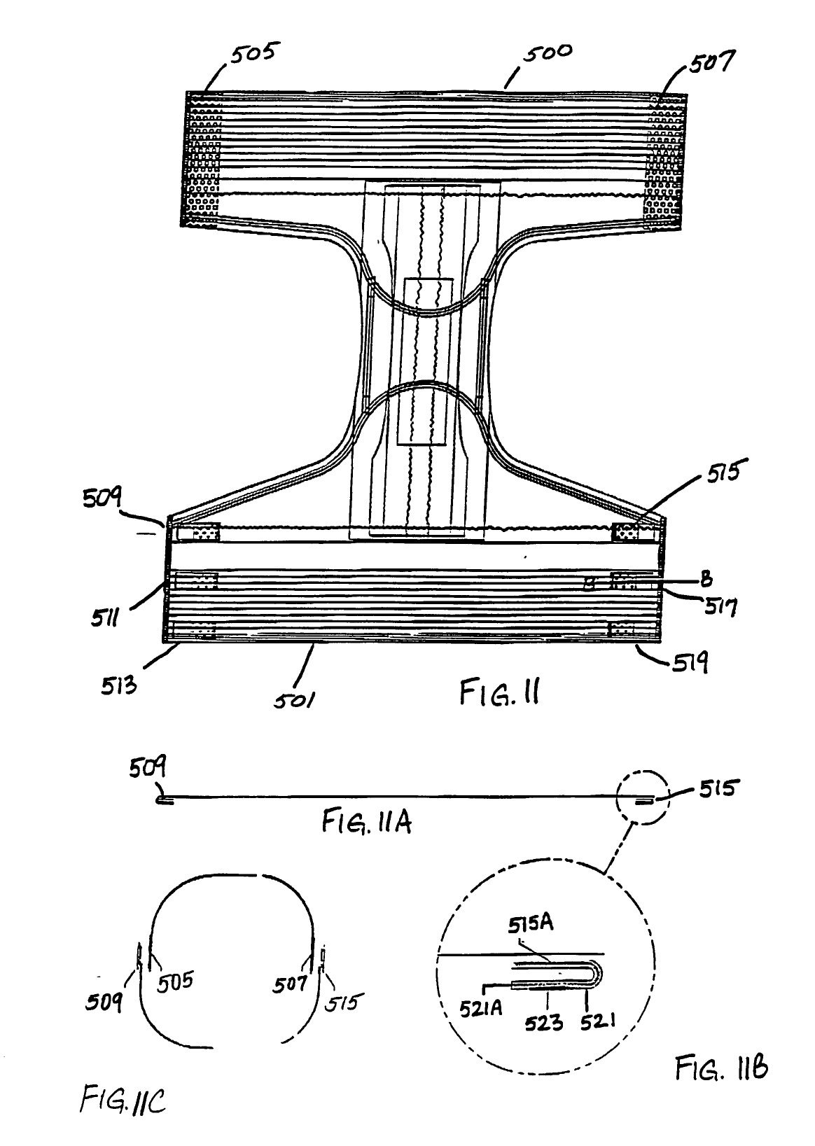

The embodiment shown in FIG. 11 is similar to the embodiment shown in FIG. 10 with that the tape tabs located on the outer surface of the back waist region 501 of the diaper 500 except that the tapes are folded as shown in FIG. 1 B. Referring to FIG. 11B which is an enlarged view of the tape tab 515, as shown therein, the tape tab construction is identical to the tapes shown in FIGS. 10, 10A, 10B and 10C. Thus, the tab has an adhesive surface 515A which is attached on the back side of the diaper and the remainder of the tape is folded to prevent the edges of the tape from interfering with the side seals. This tape also comprises a portion attached on the back side of the diaper waist, a release layer 521, a hook fastener 523 and a finger lift portion 521A.

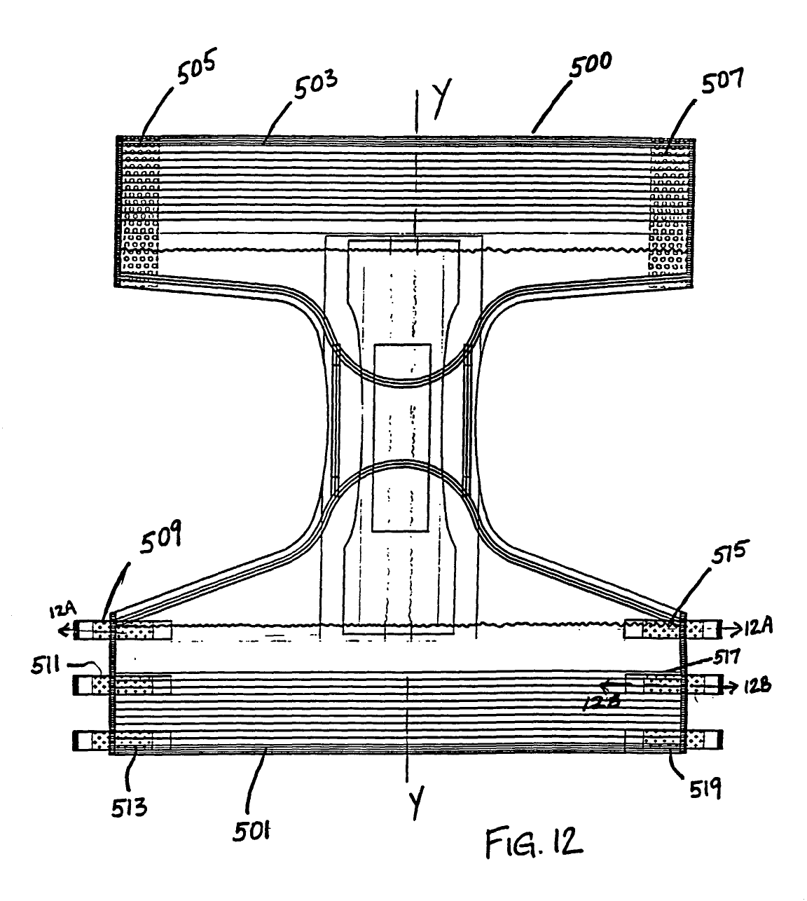

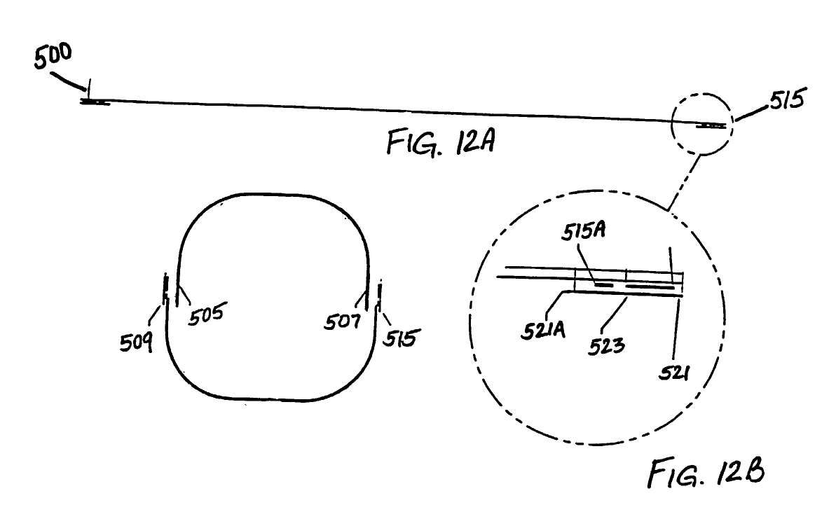

The embodiment shown in FIG. 12 is similar to the embodiment illustrated in FIG. 11 except that tape tabs attached to the back waist region project laterally beyond the edges of the respective wings. The diaper in FIG. 12 generally designated by 500 comprises a back waist region 501 having opposed lateral wings, a front waist region 503 having similar opposed lateral wings, a front waist region 503 having similar opposed lateral wings, relative to the longitudinal axis Y-Y of the diaper. The front waist region 503 comprises a pair of strips 505, 507 of loop material disposed near or at the lateral edges of the respective wings. The back waist region 501 has three tape tabs 509, 511, 513 attached thereto near the lateral edge of one of said wings, and the tape tabs 515, 517, 519 attached near or at the lateral edge of the other wing. Each of the tape tabs 509, 511, 513, 515, 517, 519 has a portion 509A, 511A, 513A, 515A, 517A and 519A, respectively, partly projecting beyond the lateral edge of each wing. These tapes are engaged with the respective loops on the back surface of the front waist region.

The manner of fastening the front and back waist regions to assemble the diaper is similar to the embodiments shown in FIG. 11. As shown in FIG. 12, if desired, side seals are provided at the respective lateral edges of each wing of the front and back waist regions. When one wishes to inspect or change the diaper, the tapes are disengaged from the loops, the side seal is torn and the diaper is inspected or changed.

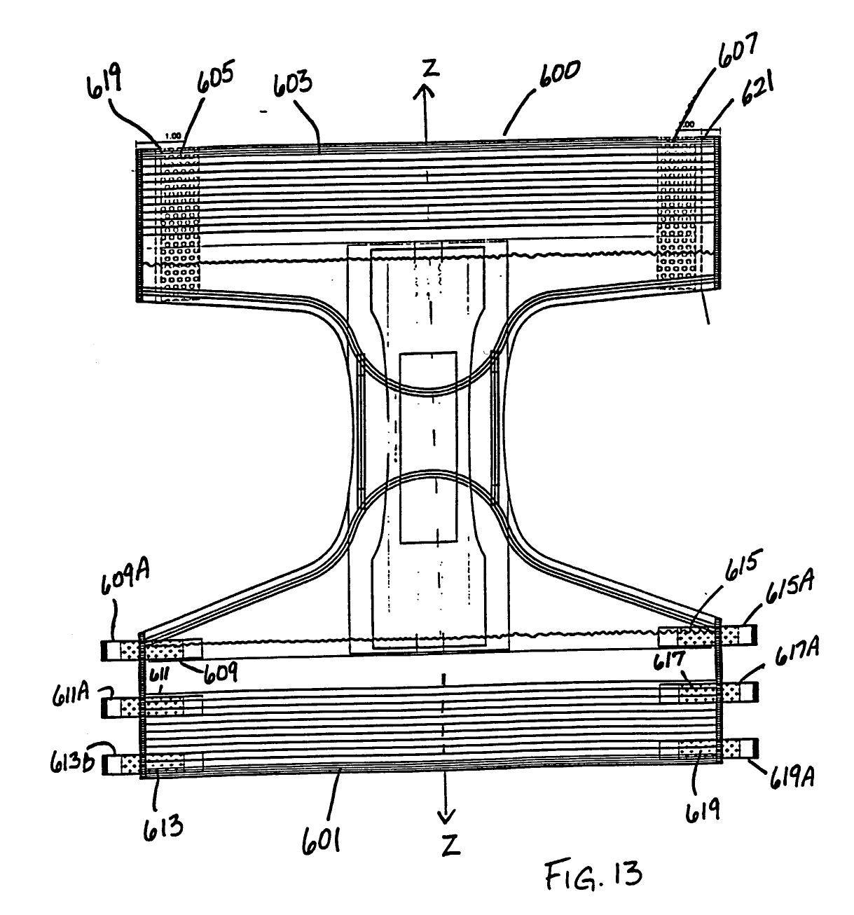

Another embodiment of the invention is illustrated in FIG. 13. The diaper shown in this figure is similar to FIG. 12 comprising a back waist region 601 having opposed lateral wings, and a front waist region 603 having similar opposed lateral wings, relative to the longitudinal axis Z-Z of the diaper. The front waist region 603 comprises a pair of strips 605,607 disposed adjacent their respective lateral edges and spaced apart therefrom. The back waist region 601 has three tape tabs 609, 611, 613 attached thereto near one lateral edge of one of said wings, and tape tabs 615, 617, 619 attached near or at the lateral edge of the other wing. The tape tabs in this embodiment are similar to the tape tabs in the embodiment shown in FIG. 12 having laterally projecting portions 609A, 611A, 613A, 615A, 617A and 619A. The difference between these two embodiments is that in the diaper shown in FIG. 13, the loop strips 605, 607 are spaced inward relative to the edges of the respective lateral wings and the front waist region 603 comprises weakened lines such as a perforated line 619 and 621 disposed adjacent each of the loop strips 605,607. Thus, when the diaper is fastened, the diaper may be inspected by tearing along the perforated lines to inspect the inside of the diaper for presence of urine or fecal material. Also, as shown in FIG. 13, the wings of the front and back waist regions have side seals for sealing the edges of the diaper.

The provision of perforated line in FIG. 13 permits tearing the diaper along the perforated lines without tearing the side seals, in order to inspect the diaper and engage the tapes with hooks to the loop surface. Each of the perforated lines may be disposed between the loop strip and the side seal or it may be disposed over the loop strip. If the diaper has perforated lines as aforesaid, the provision of side seals is optional. Whether or not the diaper is provided with side seals, the tapes with a hook surface may be engaged onto the loop strips to form the ready-to-wear diaper, and this may be performed even during the manufacture of the diaper.

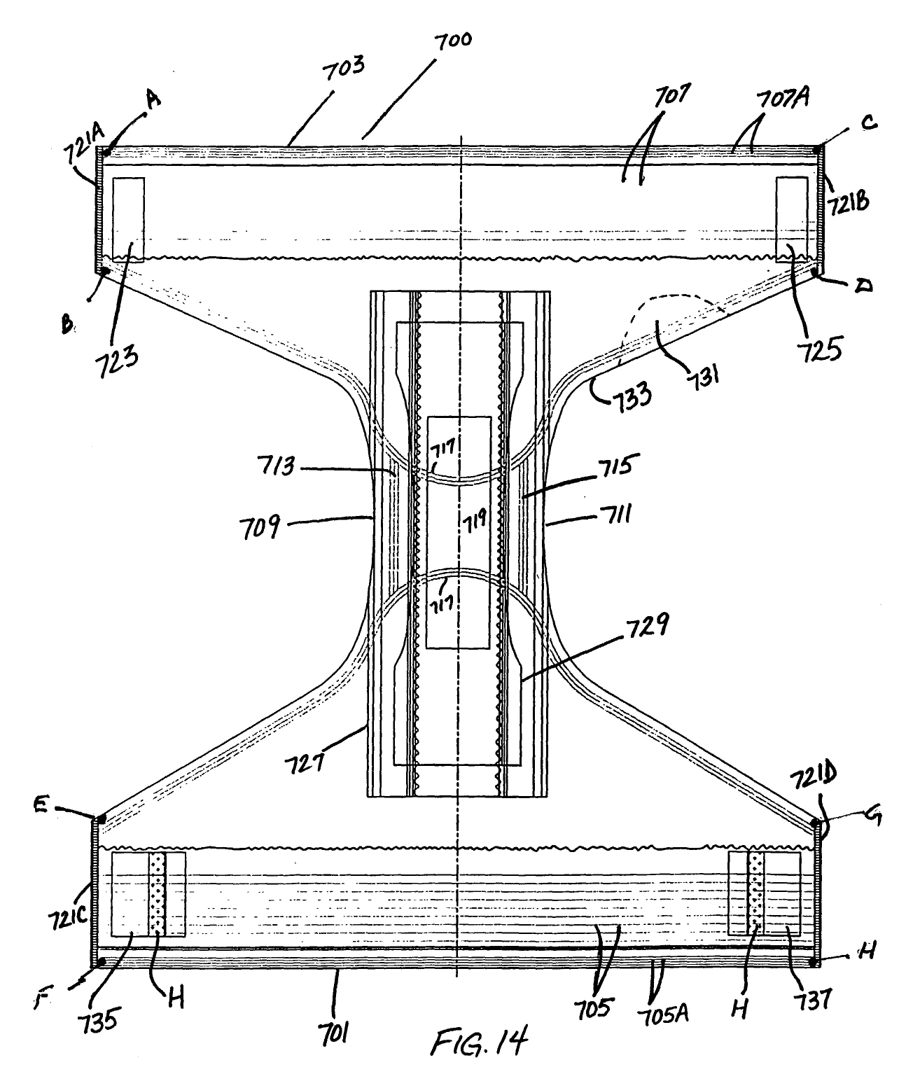

Referring to FIG. 14, there is shown a diaper generally designated by 700 in stretched view position comprising a back waist portion 701 and an elasticated front waist portion 703. Spanned across the back waist portion are a plurality of elastic elements or members 705 and belly elastic elements 705A, and a plurality of elastic elements or members 707 and belly elastic elements 707A are also spanned across the front waist portion 703. The diaper 700 also has a pair of elasticated leg openings, i.e., a right leg opening 709 and a left leg opening 711. Each of the right and left leg openings 709 and 711 is provided at its peripheral edge with a crotch elastics 713 and 715, respectively. Each leg opening also comprises thigh elastic 717 which are usually tensioned between about 0 to about 400 percent elongation, preferably between about 150 to about 250 percent elongation. The peripheral crotch elastic members 713 and 715 are tensioned between about 0 and about 400 percent elongation, preferably between about 200 and about 300 percent elongation so that the leg openings fit snugly against the crotch region 719 of the wearer in order to prevent leakage of urine or other body exudates through the leg openings. The front and back waist portions of the diaper 700 are provided with the side seals 721A,721B disposed at the outer right edges of the front waist region 703, and similar side seals 721C and 721D are disposed at the outer right edges of the back waist region 701. These side seals may be formed by heat, pressure, combination of heat and pressure, or by a suitable adhesive in a manner known in the prior art. The side seals preferably have low peel strength so that when the seals are torn or peeled away their external edges remain soft and clean. Preferably, the side seals strength may be from 1 to about 3 pounds per inch, and more preferably less than about 1.5 pound per inch. Also, a suitable material such as a nonwoven may be attached on the side seal on the back waist so that when the side seals are torn both external edges remain soft and clean.

In order to assure the mechanical integrity of the side seals, the end of each side seal, both in the front waist region and the back waist region, may be provided with a heat spot or a hot melt point such as A, B, C and D in the front waist region, and E, F, G and H in the back waist region.

The diaper 700 is shown provided with elastics at the belly portion in the front or back but such belly elastics are not strictly necessary for some diapers. An insert member 727 containing absorbent core 729 is sandwiched between the coversheet 731 and the backsheet 733.

In the embodiment illustrated in FIG. 14 the diaper construction is basically similar to the diaper shown in FIGS. 8 and 13 however, it has a different fastening system designed to further improve production and assembly of the diapers and facilitate their utilization. Thus, the diaper shown in FIG. 14 comprises a liquid permeable coversheet made of 100 percent polypropylene spunbond nonwoven which is treated with a surfactant and a backsheet made of a liquid, air and vapor impermeable polyethylene film. An absorbent core or layer is interposed between the coversheet and the backsheet. The absorbent core is made of fiberized wood pulp (fluff) containing superabsorbent polymer (SAP), preferably crosslinked polyacrylic polymer in the amount of from about 20 to about 45 weight percent of SAP based on the weight of the fiberized wood. The backsheet is placed under the absorbent layer to prevent fluid from leaking out and soiling the user's clothes or bed. Optionally, an acquisition layer may be interposed between the absorbent core and the coversheet. The acquisition layer is usually made of chemically or thermally bonded nonwoven polyester film.

The diaper shown in FIG. 14 has an elasticized back waist portion and an elasticized front waist portion, both elasticized under the same tension, with the elastic elements being attached to the outer nonwoven polypropylene, a crotch region, a belly/back portion which may comprise elastic bands attached between the outer and inner nonwoven polypropylene, under the same tension relative to each other, and an elasticated crotch region having elastic elements wherein none of the “active length” of the elastic elements intersect the through elastic. The diaper edges are sealed with side seals as hereinbefore described. Also, the belly/back elastics and the thigh elastics are usually sandwiched between the inner and outer nonwoven.

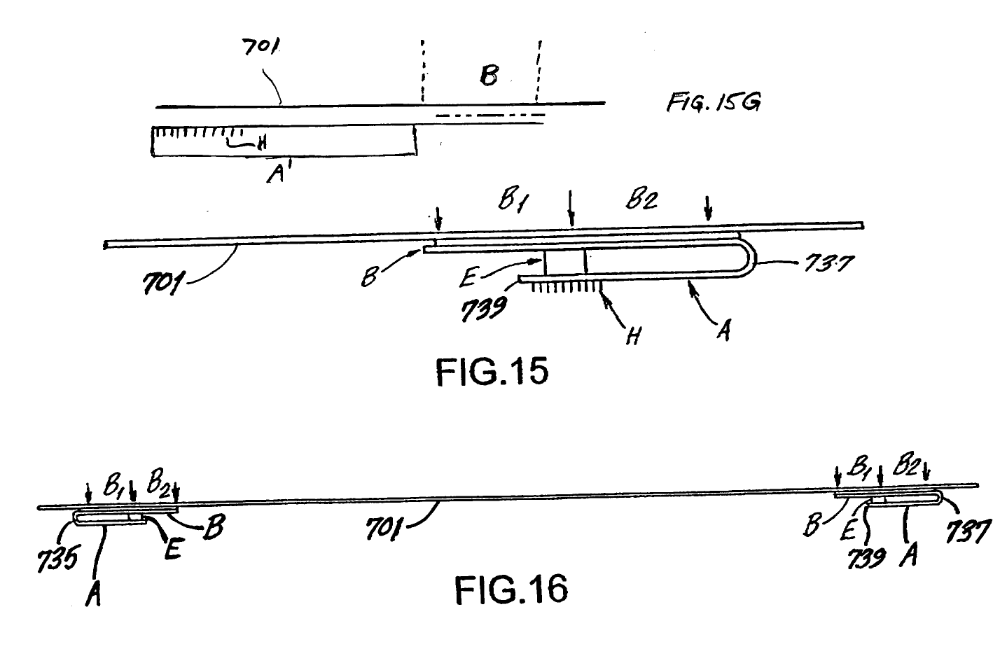

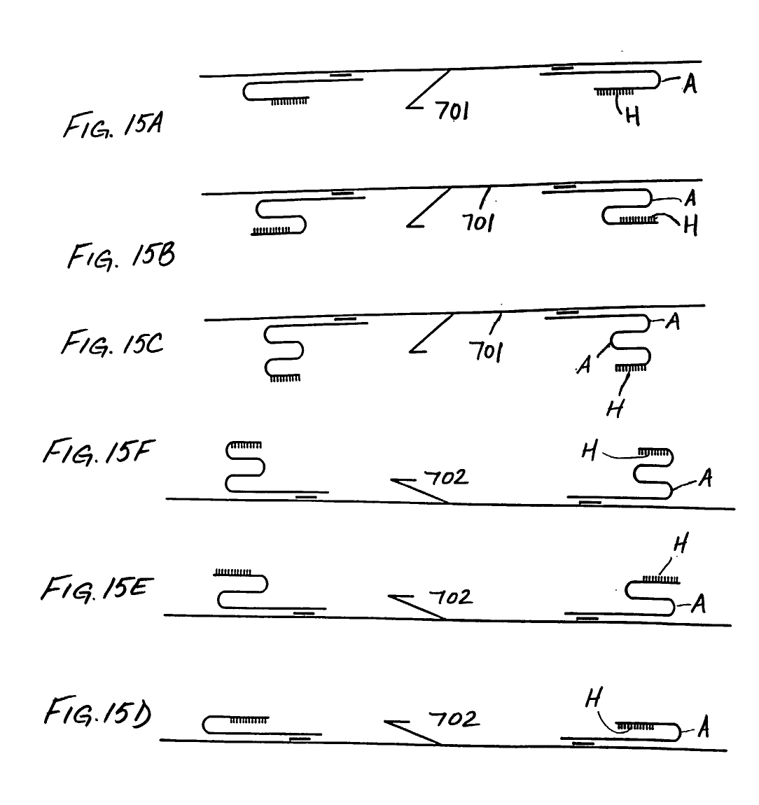

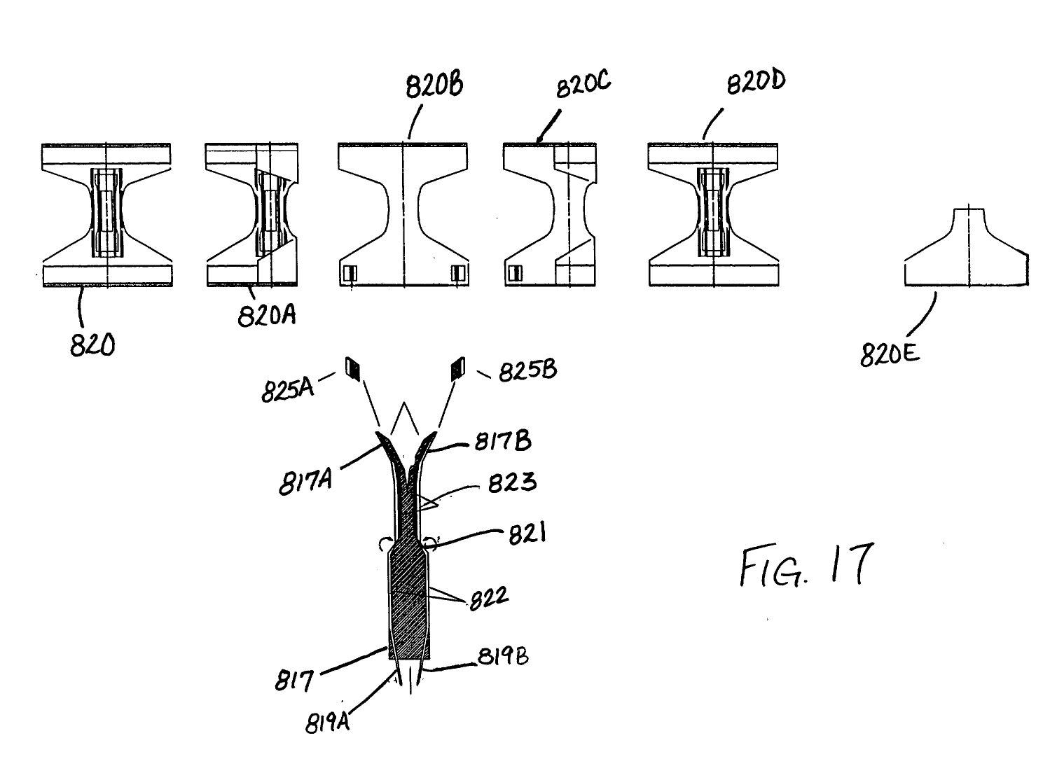

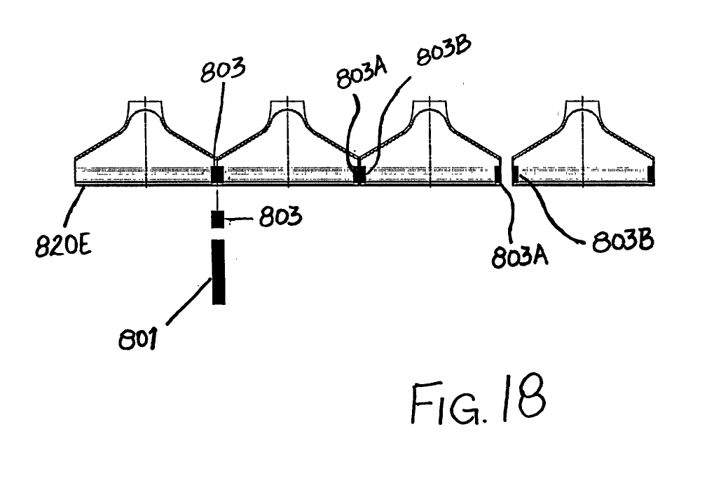

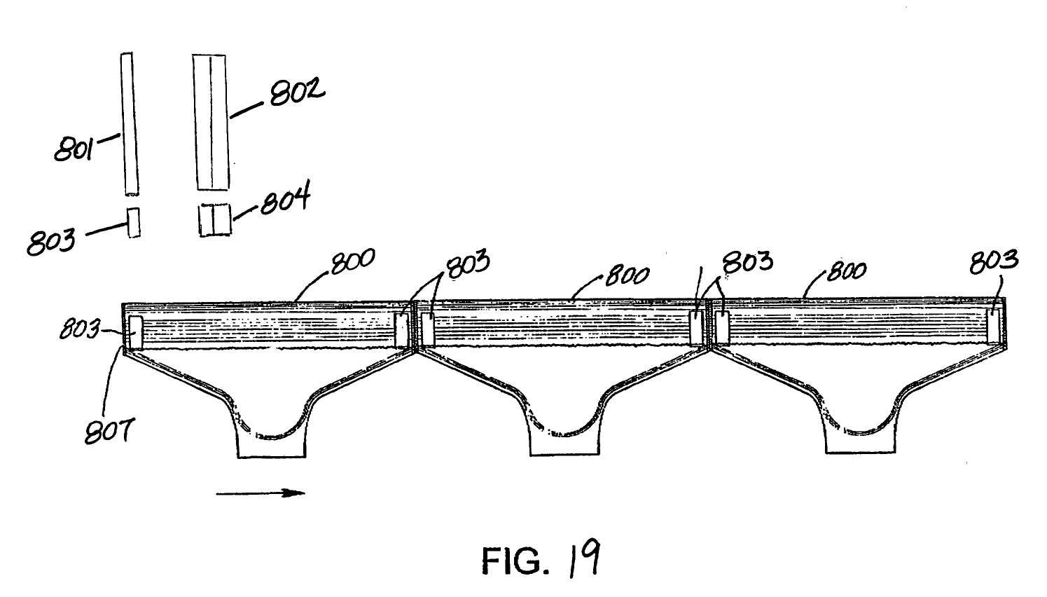

In accordance with the embodiment of the invention shown in FIGS. 14-16, the front waist portion 703 of the diaper is provided with two loop strips 723 and 725 each located at the respective lateral edges of the front waist portion adjacent the seal strips 721A and 721B. In the back waist portion 701, there are two tape tabs 735 and 737 located adjacent the seal strips 721C and 721D. The novel attachment of the tape tabs are shown in FIGS. 15 and 16. The tape tabs 735 and 737 shown in FIG. 16 are similar in construction and hence only one of them will be described in further detail. Thus, the tape tabs 737 shown in FIGS. 14-16 may be elastic or non-elastic nonwoven material comprising a fastener A portion and a portion B which is permanently secured to the outer surface of the back waist. The tab 737 may also be a composite of nonwoven elastomer-nonwoven, or nonwoven film, if desired. Alternatively, portion B may consist of a subportion B1 which is permanently secured to the outer surface of the back waist region and another subportion B2 which is releasably adhered to the outer surface back waist region. A strip of hook material H is secured to the fastener A by a suitable adhesive and a portion of the fastener A is folded as shown in FIG. 15 and the fold is maintained in place by a securement means such as a hot melt adhesive, ultrasonic bond or heat spot E. The securement mean may conveniently be a series of adhesive points of a variety of patterns, shapes and sizes, and may be aligned linearly or non-linearly.

In order to avoid contact between the hook material and the clothing of the wearer of the diaper, the fastener portion A may be folded inwardly in which case the surface of the hook will stick to the surface of the nonwoven backing. This will obviate the use of securement means, but if desired, a securement means may still be used.

Another desired construction of the fastener A is shown in FIG. 15G, which is similar to the construction illustrated in FIG. 15 except that the fastener portion A is not folded under the permanently secured subportion B1 while the product is in storage. In use, the fastener A is folded under the portion B so as to be capable of engagement with the loop strip.

The fastener A may be folded n number of times wherein n is an integer of 1 to 5 depending on location of the tape tabs and whether they are on the inner or outer surface of the back waist region. As a practical matter, however, less than 4 folds are preferred. Also, the folded portion of the fastener A terminates at a finger lift 739 (see FIGS. 15 and 16) which serves to grip the end of the fastener and lift it away from the back waist of the diaper.

As previously described in FIG. 15 the fastener A is folded once and the hook surface is secured to the fastener A away from the outer back waist surface. In FIG. 15B the fastener A is folded twice, and in FIG. 15C the fastener A is folded three times. Consequently, the hook surface H faces toward the outer surface 701 of the back waist (FIG. 15B) or away from the outer surface 701 of the back waist (FIG. 15C).

FIGS. 15D, 15E and 15F are similar to FIGS. 15A, 15B and 15C, respectively but the fastener A is attached to the inner surface 702 of the back waist, with the hook surface in each case facing in opposite direction, i.e., toward the inner surface 702 of the back waist (FIG. 15D), away from inner surface 702 of the back waist (FIG. 15C) and toward the inner surface 702 of the back waist (FIG. 15D).

The method of attachment of hook and loop fasteners will now be described with reference to FIGS. 17-19. Thus, referring first to FIG. 19, described therein is a schematic representation of the method of attaching hook strips to a pre-formed diaper chassis such as 820. In accordance with this method a web of pre-formed and interconnected web of diaper chassis 820 moves in one direction, e.g., the machine direction, is rotated 180 degrees to a new position such as at 820A in the opposite but parallel direction of the initial travel of the chassis 820 such that the back of the diaper chassis now constitutes the front of the chassis as shown in 820B. In synchronism therewith, a nonwoven web 817 from a roll of nonwoven (not shown) is moving at 90° relative to the machine direction. Also moving in the same direction as nonwoven web 817, and in spaced apart relation to each other, are the hook strips 819A, 819B, which are attached to the lower surface of the nonwoven web 817. The nonwoven web 817 with the hook strips 819A, 819B attached thereto are then folded over as at 821, are tacked down together as at 823, and the web 817 is then slit into the webs 817A and 817B. Each web 817A,817B is then turned 90° to run parallel and in the same direction as the chassis, cut to size as at 825 and the precut portions 825A,825B are then affixed to the lateral edges of the outer back waist such as shown at 820B. The web of the chassis 820B is again turned around in its initial direction of travel as in 820C and 820D where the hook strips remain attached at the outer back waist (not shown in the figure). It is understood that the hook strips 817A,817B can travel under the chassis, if desired, in the same direction as 820 and then precut to size under the chassis and affixed to the outer back waist region of the chassis. The diaper chassis with the hooks attached thereto are then folded as in 820E and the folded diaper chassis is used for attachment of the loop strips as in FIG. 18.

Referring to FIG. 18, the folded chassis diaper 820E made as hereinbefore described is advanced as a web of pre-formed, interconnected and prefolded diaper chassis, is caused to travel continuously in the machine direction during attachment of the loop strips. A web of loop material or strip 801, unwound from a roll of strip (not shown) is advanced at 90 degrees relative to the machine direction, i.e., the direction of travel of the folded chassis diaper 820E. The loop strip 801 is rotated again at 90 degrees, cut to size such as 803 and each loop strip is attached to the outer front waist region of the diaper chassis adjacent the lateral edges of two successive interconnected chassis as at 803 and 803B. The folded diaper chassis with the hook and loop fasteners attached thereto are now ready to be used.

In one aspect of the method illustrated in FIG. 18 the loop strips are affixed to the lateral edges on the outer front waist region of the chassis, the side seals are formed and the loop strip are then cut in two, such as the loop strips 803A and 803B. In another aspect of the method, the side seals may already be formed on the diaper chassis and each pre-cut loop strip is affixed over the side seal at the adjacent edges of the diaper chassis, and the loop strip is cut in two portions 803A and 803B.

Referring to FIG. 19, the method of applying the loop strips illustrated therein is similar to the method described in reference to FIG. 18 except that in FIG. 19 precut loop strips are cut to size individually so that each precut loop strip can be attached on each lateral side of the side seals at the lateral edges of the diaper front waist portion. Thus, each of two loop strips 801 and 802 is cut to desired loop strips 803,804 which are attached on each side of the side seals 807 as the diaper chassis travels in the machine direction. Thus, regardless of how the loop strips are attached the lateral edges of the front waist of the diaper chassis will be provided with loop strips which, and will engage with complementary hooks attached to the lateral edges of the back waist portion of the diaper as hereinbefore described.

It must be mentioned that in the construction of a diaper chassis, the diaper may be preformed, if desired, with the outer surface of its front waist region made of loop material. In this construction, the step of attachment of the loop strips is omitted since the diaper chassis with the hooks attached thereto will engage with the loop surface itself, i.e., the outer surface of the front waist region, without the need for the step of attaching individual precut loops to the diaper chassis.

While the diapers of this invention and their method of manufacture have both been described with certain degrees of particularity, some changes and modifications may be made therein which are obvious to those skilled in the art from the foregoing description. For example in FIG. 17 two separate webs of hook fasteners may be used. This means that two separate hook applicators may be used rather than only one applicator shown in FIG. 17. Also, the nonwoven web may be pre-coated with adhesive or it may be coated with an adhesive in line. These and other changes are obvious and within the scope of the present invention.

1. An integral pull-up type absorbent article comprising:
(a) a coversheet;
(b) a backsheet;
(c) an absorbent layer between said coversheet and said backsheet;
(d) a pair of spaced apart leg openings;
(e) a waist region comprising a front waist portion and a back waist portion, each of said portions having a laterally extending portion terminating in a lateral side edge,
(f) a first strip of loop material on one of said lateral edges and a second strip of loop material adjacent the other one of said lateral edges, on said front waist region; and
(g) a first tape tab adjacent one of said lateral edges and a second tape tab adjacent the other one of said lateral edges, on said back waist region,
wherein each of said tape tabs comprises a first portion permanently secured to said back waist portion, and a fastener portion spaced apart relative to said permanently attached first portion, said fastener portion being folded under the first portion of said tape tab and is secured thereto by a securement means, wherein said folded portion comprises hook material secured to said folded portion, and wherein when said back waist portion is folded over said front waist portion, each of said hooks engages a correspondingly aligned loop strip.

2. An integral pull-up absorbent article as in claim 1 wherein said first tape tab comprises a subportion permanently attached to said back waist portion, and another subportion releasably adhered to said back waist portion.

3. An integral pull-up absorbent article as in claim 1 wherein each of said lateral edges includes a side seal.

4. An integral pull-up absorbent article as in claim 2 wherein each of said lateral edges includes a side seal.

5. An integral pull-up absorbent article as in claim 1 wherein said folded portion has a free end provided with a finger lift portion adapted to be gripped by the fingers and lifted to lift away the fastener from said back waist portion.

6. An integral pull-up absorbent article as in claim 2 wherein said folded portion has a free end provided with a finger lift portion adapted to be gripped by the fingers and lifted to lift away the fastener from said back waist portion.

7. An integral pull-up absorbent article as in claim 3 wherein said folded portion has a free end provided with a finger lift portion adapted to be gripped by the fingers and lifted to lift away the fastener from said back waist portion.

8. An integral pull-up absorbent article as in claim 4 wherein said folded portion has a free end provided with a finger lift portion adapted to be gripped by the fingers and lifted to lift away the fastener from said back waist portion.

9. An integral pull-up absorbent article as in claim 3 wherein each of said side seals is adhesively secured to its respective lateral edge.

10. An integral pull-up absorbent article as in claim 4 wherein each of said side seals is adhesively secured to its respective lateral edge.

11. An integral pull-up absorbent article as in claim 5 wherein each of said lateral edges comprises a side seal.

12. An integral pull-up absorbent article as in claim 6 wherein each of said lateral edges comprises a side seal.

13. An integral pull-up absorbent article as in claim 7 wherein each of said lateral edges comprises a side seal.

14. An integral pull-up absorbent article as in claim 8 wherein each of said lateral edges comprises a side seal.

15. An integral pull-up absorbent article as in claim 11 wherein each of said side seals is adhesively secured to its respective lateral edge.

16. An integral pull-up absorbent article as in claim 12 wherein each of said side seals is adhesively secured to its respective lateral edge.

17. An integral pull-up absorbent article as in claim 13 wherein each of said side seals is adhesively secured to its respective lateral edge.

18. An integral pull-up absorbent article as in claim 14 wherein each of said side seals is adhesively secured to its respective lateral edge.

19. An integral pull-up absorbent article as in claims 1, 2, 3, 4, 5, 6, 7, 8, 9, 10, 11, 12, 13, 14, 15, 16, 17 or 18 wherein said securement means is hot melt adhesive or ultrasonic bond.

20. An integral pull-up absorbent article as in claim 19 wherein each lateral wing of said front waist region comprises a weakened line.

21. An integral pull-up absorbent article as in claim 20 wherein each of said weakened lines is disposed adjacent a loop strip.

22. An integral pull-up absorbent article as in claim 20 wherein each of said weakened line is disposed on a loop strip.

23. An integral pull-up type absorbent article comprising:
(a) a coversheet;
(b) a backsheet;
(c) an absorbent layer between said coversheet and said backsheet;
(d) a pair of spaced apart leg openings;
(e) a waist region comprising a front waist portion and a back waist portion, each of said portions having a laterally extending portion terminating in a lateral side edge, wherein said front waist portion has an outer surface made of a loop material, and
(f) a first tape tab adjacent one of said lateral edges and a second tape tab adjacent the other one of said lateral edges, on said back waist region,
wherein each of said tape tabs comprises a first portion permanently secured to said back waist portion, and a fastener portion spaced apart relative to said permanently attached first portion, said fastener portion being folded under the first portion of said tape tab and is secured thereto by a securement means, wherein said folded portion comprises hook material secured to said folded portion, and wherein when said back waist portion is folded over said front waist portion, each of said hooks engages a portion of the loop surface on said front waist portion.

24. An integral pull-up type absorbent article as in claim 1 wherein said fastener portion is folded inwardly thereby avoiding contact between the hook fastener and the wearer.

25. An integral pull-up type absorbent article as in claim 23 wherein said fastener portion is folded inwardly avoiding contact between the hook fastener and the wearer.

26. An integral pull-up type absorbent article as in claim 1 wherein each lateral edge of said front waist region comprises side seals and each lateral edge of said back waist region comprises side seals and wherein a strip of nonwoven is attached to the side seal on the back waist region.

27. An integral pull-up type absorbent article as in claim 23 wherein each lateral edge of said front waist region comprises side seals and each lateral edge of said back waist region comprises side seals and wherein a strip of nonwoven is attached to the side seal on the back waist region.

28. A method of attaching hook and loop fasteners to a pull-up type absorbent article wherein said article comprises a chassis having a front waist region and a back waist region, each of said regions having opposite laterally extending portions each terminating in a lateral edge, said method comprising:
(a) advancing a web of preformed interconnected chassis in one direction,
(b) rotating said web of pre-formed interconnected chassis 180 degrees and continuing to advance said web,
(c) advancing a nonwoven web in 90 degrees direction relative to the direction of travel of said rotated chassis web, said nonwoven web having a lower surface and a top surface, said lower surface having hook fasteners attached thereto,
(d) bifurcating said nonwoven web into two separate webs, turning each separated web 90 degrees to run parallel to and in the same direction as the chassis, cutting each of said separate webs into individual predetermined sizes, and affixing each of said portions to a lateral edge of the outer back of each of said advancing chassis,
(e) rotating the resulting chassis again 180 degrees and folding said chassis,
(f) advancing the web of said folded chassis in the same direction,
(g) advancing a strip of loop material at 90 degrees relative to the direction of travel of said folded chassis web, severing said loop strip to individual predetermined portions and attaching each of said severed loop strips to the outer front waist region of the lateral edges of successive interconnected chassis.

29. A method of attaching hook and loop fasteners to a pull-up type absorbent article wherein said article comprises a chassis having a front waist region and a back waist region, each of said regions having opposite laterally extending portions each terminating in a lateral edge, said method comprising:
(a) advancing a web of pre-formed interconnected chassis in one direction,
(b) rotating said web of pre-formed interconnected chassis 180 degrees and continuing to advance said web,
(c) advancing a nonwoven web in 90 degrees direction relative to the direction of travel of said rotated chassis web, said nonwoven web having a lower surface and a top surface, said lower surface having hook fasteners attached thereto,
(d) bifurcating said nonwoven web into two separate webs, turning each separated web 90 degrees to run parallel to and in the same direction as the chassis, cutting each of said separate webs into individual predetermined sizes, and affixing each of said portions to a lateral edge of the outer back of each of said advancing chassis,
(e) rotating the resulting chassis again 180 degrees and folding said chassis,
(f) advancing the web of said folded chassis in the same direction,
(g) advancing a strip of loop material at 90 degrees relative to the direction of travel of said folded chassis web, severing said loop strip to individual predetermined sizes and attaching each of said severed loop strips to the outer front waist region of the lateral edges of successive interconnected chassis.

30. A method of attaching hook and loop fasteners to a pull-up type absorbent article wherein said article comprises a chassis having a front waist region and a back waist region, each of said regions having opposite laterally extending portions each terminating in a lateral edge, said method comprising:
(a) advancing a web of pre-formed interconnected chassis in one direction,
(b) rotating said web of pre-formed interconnected chassis 180 degrees and continuing to advance said web,
(c) advancing a nonwoven web in 90 degrees direction relative to the direction of travel of said rotated chassis web, said nonwoven web having a lower surface and a top surface,
(d) advancing two separate webs of hook fasteners in the same direction as said nonwoven web and attaching said web of hook fasteners to the lower surface of said nonwoven web, folding said nonwoven hook web, tacking said nonwoven web and said hook web together, bifurcating same into two separate composite web of nonwoven/hook fastener, turning each of said separated composite web 90 degrees to run parallel to and in the same direction as the chassis, cutting said composite web into individual predetermined sizes, and affixing each of said precut portions to a lateral edge of the outer back of each of said advancing chassis,
(e) advancing a strip of loop material at 90 degrees relative to the direction of travel of said folded chassis web, severing said loop strip to individual predetermined sizes and attaching each of said severed loop strips to the outer front waist region of the lateral edges of successive interconnected chassis.

31. A method of making a pull-up type absorbent article wherein said article comprises a chassis having a front waist region and a back waist region, each of said regions having opposite laterally extending portions each terminating in a lateral edge, and wherein said front waist region has an outer surface made of a loop material, said method comprising:
(a) advancing a web of pre-formed interconnected chassis in one direction,
(b) rotating said web of pre-formed interconnected chassis 180 degrees and continuing to advance said web,
(c) advancing a nonwoven web in 90 degrees direction relative to the direction of travel of said rotated chassis web, said nonwoven web having a lower surface and a top surface, said lower surface having hook fasteners attached thereto,
(d) bifurcating said nonwoven web into two separate webs turning each of said separated webs 90 degrees to run parallel to and in the same direction as the chassis, cutting each of said separate webs into individual predetermined sizes, and affixing each of said portions to a lateral edge of the outer back of each of said advancing chassis,
(e) engaging said hook fastener to said loop surface on the outer waist region of said chassis.

32. A method of making a pull-up type absorbent article wherein said article comprises a chassis having a front waist region and a back waist region, each of said regions having opposite, laterally extending portions each terminating in a lateral edge, and wherein said front waist regions has an outer surface made of a loop material, said method comprising:
(a) advancing a web of pre-formed interconnected chassis in one direction,
(b) rotating said web of pre-formed interconnected chassis 180 degrees and continuing to advance said web,
(c) advancing a nonwoven web in 90 degrees direction relative to the direction of travel of said rotated chassis web, said nonwoven web having a lower surface and a top surface,
(d) advancing two separate webs of hook fasteners in the same direction as said nonwoven web and attaching said web of hook fasteners to the lower surface of said nonwoven web, folding said nonwoven hook web, tacking said nonwoven web and said hook web together and bifurcating same into two separate composite web of nonwoven/hook fastener, cutting said composite web into individual portions of predetermined sizes, rotating said precut portion 90 degrees and affixing each of said precut portions to a lateral edge of the outer back of each of said advancing chassis, and
(e) engaging said hook fasteners with said loop surface on said front waist region of said diaper chassis.

33. Integral pull-up type absorbent article comprising:
(a) a coversheet;
(b) a backsheet;
(c) an absorbent layer between said coversheet and said backsheet;
(d) a pair of spaced apart leg openings;
(e) a waist region comprising a front waist portion and a back waist portion, each of said portions having a laterally extending portion terminating in a lateral side edge,
(f) a first strip of loop material on one of said lateral edges and a second strip of loop material adjacent the other one of said lateral edges, on said front waist region; and
(g) a first tape tab adjacent one of said lateral edges and a second tape tab adjacent the other one of said lateral edges, on said back waist region,
wherein each of said tape tabs comprises a first portion permanently secured to said back waist portion, and a fastener portion spaced apart relative to said permanently attached first portion, wherein said fastener portion comprises hook material secured to said folded portion, and wherein when said back waist portion is folded over said front waist portion, each of said hooks engages a correspondingly aligned loop strip.
